(19) United States
(12) Patent Application Publication
(10) Pub. No.: US 2015/0066143 A1
(43) Pub. Date: Mar. 5, 2015

(54) Silicone Filamentous Configurations

(71) Applicants: Vishnu Rumalla, Southlake, TX (US); Donna K. Jackson, Fort Worth, TX (US)

(72) Inventors: Vishnu Rumalla, Southlake, TX (US); Donna K. Jackson, Fort Worth, TX (US)

(21) Appl. No.: 14/472,766

(22) Filed: Aug. 29, 2014

Related U.S. Application Data
(60) Provisional application No. 61/872,052, filed on Aug. 30, 2013.

(51) Int. Cl. A61F 2/12; A61L 27/50; B29C 44/20; A61L 27/02

(52) U.S. Cl. CPC A61F 2/12; A61L 27/025; A61L 27/50; B29C 44/20; A61L 2400/16; B29K 2105/046
USPC 623/8; 264/54

(57) ABSTRACT

This novel solid silicone device is designed to let women have more and better choices in breast implant technology for both cosmetic and reconstructive purposes, include shape filling and solid devices overcoming longstanding needs.

### CROSS-REFERENCE TO RELATED APPLICATIONS

This application claims the priority benefit of U.S. Patent Application 61/872,052 filed Aug. 30, 2013. This application fully incorporates by reference, U.S. Pat. No. 8,501,645, along with U.S. patent application Ser. Nos. 11/171,900, 12/906,819, and 13/880,138, each as if fully set forth herein in their entirety along with U.S. Patent Publication US 2014/0199915.

### BACKGROUND OF THE DISCLOSURE

1. Field of the invention

This invention relates to the field of prosthetic implants. More specifically, the invention comprises a novel silicone filler of continuous, woven, connected fibers encapsulated into solid or other such materials which optionally replaces air, silicon gels, liquids, liquid/gel/air combinations and the like. Likewise, additional processing using nitrogen has yielded improved specific gravity of resulting products by subject processes.

2. Objects and Summary of the Disclosure

Briefly stated, Silicone breast implants have been commercially available for decades. The first generation of these devices was made of a liquid silicone. Unfortunately, these devices had a high rupture rate, potential silicone migration and were removed from the market in the 1980s. A high profile lawsuit led leaders in plastic surgery and industry to make a conscious effort to develop a superior breast implant product. By 2007, the FDA approved the most recent breast implant which is infused with a cohesive gel technology. The current devices have enjoyed a good safety and performance record but have several limitations which can be improved upon. First, these devices are very heavy. The weight of these devices has been related to symptoms like back and neck pain and mechanical complications like “bottoming out” of the implant. In addition, long incisions are required to place these implants. Finally, the implants are not adjustable making it difficult to deal with breast asymmetries.

According to embodiments, there is disclosed a novel enhanced implant system, which comprises, in combination, at least a silicon filler of continuous, woven, connected fibers encapsulated into solid or other such materials which into solid or other such materials which replace air, silicon gels, liquids, liquid/gel/air combinations, wherein nitrogen in introduced into the material.

According to embodiments, there is disclosed a novel enhanced implant system, further comprising an external solid silicone shell filled with a web/pattern of continuous inner-connected woven silicone fibers, including nitrogen infused fibers.

According to embodiments, there is disclosed a novel enhanced implant system, further comprising silicone filamentous fiber using medical grade silicone and generating a continuous flow of connective, cured, silicone fibers end products which are significantly decreased in weight, and have a preferred specific gravity.

According to embodiments, there is disclosed a novel enhanced implant system, being a true solid and a continuous strand which reduces and/or eliminates migration.

According to embodiments, there is disclosed a novel enhanced implant system, further comprising the ability of the connected fiber strands, to retain their originally designed shape when compressed to significantly aid in reducing incision size.

According to embodiments, there is disclosed a system in at least a second form consisting of one variety amongst many valves which allows saline to be injected to adjust volume and fill, which will allow greater opportunity to correct breast asymmetry.

According to embodiments, there is disclosed a process used in breast augmentation, breast reconstruction, and mastopexy (breast lift), and any other procedures relevant, using silicone fibers pull-truded and nitrogen infused to confer a preferred specific gravity.

### BRIEF DESCRIPTION OF THE FIGURE

FIG. 1 schematically depicts a flow-charted summary of steps according to the instant disclosure for decreasing specific gravity of a Filamentous Silicone Fiber (Fil-Sil) according to the present invention.

### BRIEF SUMMARY OF THE DISCLOSURES

To address the shortcoming listed, new silicone breast implant medical devices are offered for consideration. Subject devices consist of an external solid silicone shell and filled with a web/pattern of continuous inner connected woven silicone fibers. This silicone filamentous fiber is used to fabricate end products which are significantly improved. By using medical grade silicone and generating a continuous flow of connective, cured, silicone fibers end products are significantly decreased in weight. As a true solid and a continuous strand it should reduce and/or eliminate migration. The ability of the connected fiber strands, to retain their originally designed shape when compressed will significantly aid in reducing incision size.

While it is considered there are multiple combinations of applications using said filamentous fibers, the initial implants are suited in two forms. The first form is pre-filled in the manufacturing process. The second form consists of one variety amongst many valves which allows saline to be injected to adjust volume and fill, which will allow greater opportunity to correct breast asymmetry. These new and novel implantable medical devices are used in breast augmentation, breast reconstruction, and mastopexy (breast lift), among other things.

This novel solid silicone device is designed to let women have more and better choices in breast implant technology for both cosmetic and reconstructive purposes.

### DETAILED DESCRIPTION OF THE DISCLOSURE

Filamentous Silicone Fibers (“Fil-Sil”) are defined as silicone fibers within the scope of the process defined by U.S. Pat. No. 8,501,645 wherein decreased specific gravity results from a blowing agent being used to refine a closed cell structure with a non-porous skin. Artisans readily understand that this makes scalable improvements and material improvements which were unexpected at the time the prosthetics claims herein were being developed.

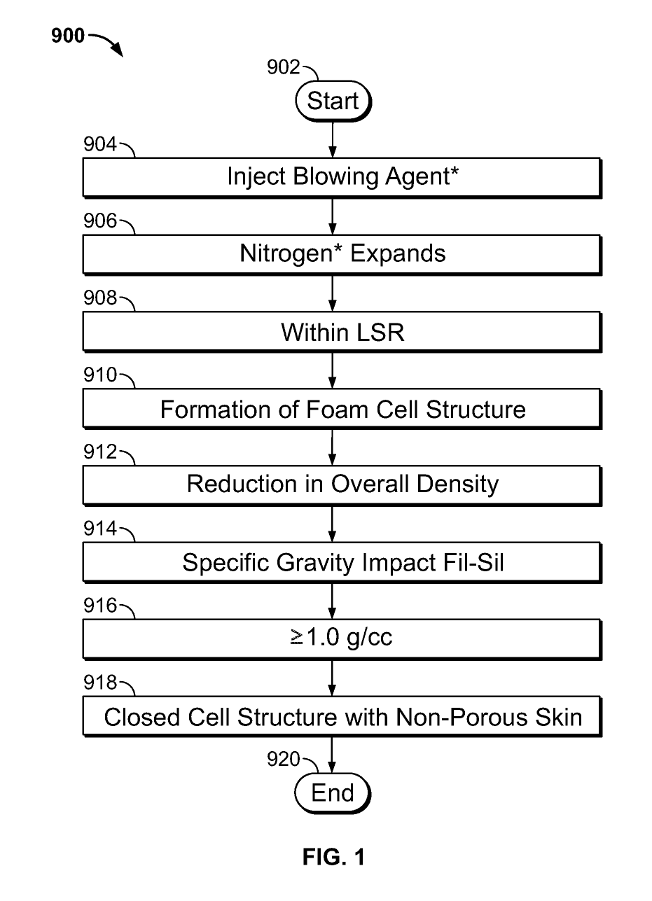

Referring now to FIG. 1, process 900 involved injecting a blowing agent 904, which may be nitrogen 906 which expands with LSR 908 to form a foam cell structure 910, having lower density 912, producing a lower specific gravity product 914, 918 with, for examples a specific gravity equal to or below 1.0 g/cc.

The present inventors have addressed a longstanding need, namely generating prosthetics using silicone whereby the specific gravity is improved using nitrogen. Artisans readily understand the nature and extent of the instant system to be useful for breast prosthetics and related desiderata.

### EXAMPLE 1

A silicone filament is created from a combination of conventional silicone precursor elements as known to those skilled in the art from liquid silicone rubber parts A (catalyst: reinforced dimethyl methylvinyl siloxanes), Part B (crosslinking agent: reinforced dimethyl methylhydrogen) (For example, Rhodia-A LSR-4330 Silbione® HCA Part #V50131A-40 Lot 0409031, B LSR-10 Silbione® HC Part #V500004B-40 Lot 26776, Medical Grade available from Rhodia Europe/Rhodia SA, 26, quai Alphonse Le Gallo, 92512 Boulogne-Billancourt cedex and Rhodia Amerique du Nord/Rhodia Inc., 259 Prospect Plains Road, Conn. 7500, Cranbruy N.J. 08512-7500) which is then whipped, beaten and extruded through a die according to the teachings of the present disclosure. Likewise heat cured silicone rubbers are commercially available in 2 parts systems (A and B). These parts need to be precisely mixed to produce consistent product, requiring controlled pumping and mixing equipment. In 2004 Laur Silicone developed a ONE PART® heat curable liquid silicone rubber using the EASY CURE® technology. This EASY CURE® system greatly reduces the requirements for meter mix equipment. ONE PART® liquid silicone can be supplied fully compounded and ready to use.

Extruding nozzles or screen assemblies and die plates may alternately be employed having aperture configurations which drive aspects of the specific tubular orientation desired to be achieved. Artisans understand that liquid silicone materials will not be altered in terms of physical properties until adequate temperatures are achieved, for example (once A and B are mixed, these materials have short pot lives that are inversely related to temperature) and the instant disclosure has used a heating chamber at 350 degrees Fahrenheit which circulates hot air to house the extrudate for drying.

Fabricators typically operate at vulcanization temperatures between 250 degrees and 375 degrees Fahrenheit. Actual results will depend on the design and size. According to this example, as the elongated filaments are extruded through the apertures or screen elements long strands hang curtain-like downward.

The curtain-like strands or threads, filaments, fibers, twines may be extruded directly from the die plate into the drying chamber, for example, the curtain-like group of filaments is deposited directly on a metal mesh that prevents sticking and allows for easy removal. The metal mesh itself is mounted on a moving belt that pushes the filaments outside of the heating chamber for easy removal, where they are reeled on a cocoon-like holding rod. A resulting product has diameter that varies between a few and several millimeters in diameter.

### EXAMPLE 2

The process steps set forth above in the first example apply, however the diameters achieved are larger, here silicone fibers may be created also by mixing the silicone emulsion combining at least one of the ratios selected from the group consisting of 30/70, 40/60 and substantially equal aliquots of the two different liquid silicon precursors, of silicone A and B, medical grade (available also from Applied Silicone, Liquid Silicone Rubber, LSR 10, Part A, Lot 17689, and Silastic® Q7-4850 Lot hh 063161; Part B Silastic® Q7-48750 medical grade liquid silicone rubber). Medical grade silicone ingredients yield biocompatible product that may be used to fill containers used, for example for cosmetic facial enhancements, or as breast implants without any concerns about leakage, biocompatability, contamination, or many of the other problems which currently exist. The process steps for the second example are similar to Example I, and different from the prior art by using a metal die, which is in its most rudimentary form simply a metal disc with apertures extending through it, following at least about ten minutes of mechanical agitation which suspends air into the mixture, during which time nitrogen is introduced into the mixture.

Strands of silicone having a larger diameter may also be mixed from at least one of the Dow Corning® brand of constituents, intermediates from GE Silicones® or the Walker-Chemie® brands of products. The silicone filaments, fibers and strands have elastic memory, tensil strength and a sufficiently high fill density in a compacted state rendering them effective for occupying a predetermined space over time without degrading, the diameter of the objects of this example are greater than several millimeters in diameter.

### EXAMPLE 3

As discussed above, a silicone emulsion was made by combining at least one of the ratios selected from the group consisting of 30/70, 40/60 and substantially equal aliquots of the two different liquid silicon precursors, substantially equal aliquots of silicones A and B, medical grade selected from the group consisting of 10, 20, 30, 40 and 50 type (manufacturer's suggestion for second-wise definitions of the optimal curing time for crosslinking to be achieved). The sources are as discussed in the first two examples. Mechanical agitations using a motor driven mixing machine were imparted to the mixture for approximately ten minutes. During this time nitrogen has been introduced into the mixture, having an impact on resulting specific gravity.

The fluffed emulsion was then moved into a pressing machine having a die plate having a plurality of apertures through which it was extruded onto a rotating deposit plate into a drying chamber at 350 degrees Fahrenheit. The resulting product was chopped into pieces, as discussed above, curtain-like strands may be extruded directly from the die plate into the drying chamber, for example, the curtain-like group of filaments is deposited directly on a metal mesh that prevents sticking and allows for easy removal. The metal mesh itself is mounted on a moving belt.

Prior to the static mixer, a blowing agent will be injected into the Liquid Silicone Rubber (LSR) being used to produce Fil-Sil. In the current equipment setup, this will be done at the injection port, commonly used to inject pigment, at the “T” joint where LSR Part “A” and LSR Part “B” unite. The injection port includes a check valve to prevent LSR from flowing out of the port. The blowing agent can be in the form of an inert gas, water, or a chemical blowing agent.

Gas is injected under pressure and then expands within the LSR as it exits the extrusion die, forming a foam cell structure in the LSR, which reduces the overall density.

Water is commonly used as a blowing agent due to the rapid expansion that results when heated water turns into steam. With water, it is the heat introduced by the oven, rather than the rapid decrease in pressure at the face of the extrusion die, that causes the foam cell structure to form.

Chemical blowing agents can utilize chemical reactions initiated by the heat of the oven or rapid decompression to facilitate the formation of the foam cell structure.

In all three cases, a closed cell structure with a non-porous skin is desired. Please note that an open cell structure would be counter-productive in the application you had described since fluid surrounding the Fil-Sil would infiltrate the fibers and fill in the voids, thus negating any benefits to lowering the Specific Gravity. The extent to which the Specific Gravity of the Fil-Sil is lowered will depend heavily on the type and quantity of blowing agent utilized.

The risk of such an endeavor is that the tiny, rapid expansions caused by the blowing agent could potentially weaken the fibers to the point that they break prior to curing in the infrared oven, which would cause knots and clumps in the Fil-Sil.

We worked with Nitrogen first because it would cause the smallest change to Specific Gravity. Goal is value for specific gravity below 1.0 g/cc. For this option, we would hook up a tube from a Nitrogen canister to the pigment injection port.

Water would be the next easiest blowing agent to introduce through the pressurized color pot. Steam will expand to about 1,000 times the original volume of water, but this reaction would occur a little further downstream in the oven instead of at the face of the extrusion die, which could help prevent breakages.

While the method and apparatus have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Merriam-Webster's Unabridged Dictionary, the latest edition of which is hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to United States patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A novel enhanced implant system, which comprises, in combination: at least a silicon filler of continuous, woven, connected fibers encapsulated into solid or other such materials which into solid or other such materials which replace air, silicon gels, liquids, liquid/gel/air and combinations thereof.

2. A system according to claim 1, further comprising an external solid silicone shell filled with a web/pattern of continuous inner-connected woven silicone fibers, having an improved specific gravity enhanced by nitrogen infusion.

3. A system according to claim 2, further comprising silicone filamentous fiber using medical grade silicone and generating a continuous flow of connective, cured, silicone fibers end products which are significantly decreased in weight, while having a specific gravity enhanced by nitrogen infusion.

4. The system claim 3, being a true solid and a continuous strand which reduces and/or eliminates migration.

5. The system of claim 4, further comprising, in combination: elastic memory, namely the ability of the connected fiber strands, to retain their originally designed shape when compressed to significantly aid in reducing incision size.

6. Improved Filamentous Silicone Fiber (Fil-Sil), further comprising, in combination:
at least a second form consisting of one variety amongst many valves which allows saline to be injected to adjust volume and fill, which will allow greater opportunity to correct breast asymmetry.

7. The Improved Fil-Sil, of claim 6 used in breast augmentation, breast reconstruction, and mastopexy (breast lift), and any related procedures.

8. The Improved Fil-Sil, of claim 7, further comprising silicone having nitrogen incorporated therein.

9. The Improved Fil-Sil, of claim 8, wherein said nitrogen housing silicone has a specific gravity lowered to at least about 1.0 g/cc.

10. The Improved Fil-Sil, of claim 9, wherein said nitrogen housing silicone has a specific gravity changing between at least 0.70 g/cc and 0.90 g/cc.

11. A process for enhancing Filamentous Silicone Fibers (Fil-Sil), which comprises in combination:
injecting a blowing agent, such as nitrogen, into a Liquid Silicone Precusor;
expanding said blowing agent with the Liquid Silicone Rubber Precursor therein;
forming of foam cell structures;
reducing overall density of the mixture;
impacting specific gravity of a resultory mixture; and
generating closed cell structures having non-porous skin having desired specific gravity ranges.

12. Products, produced by the process of claim 11, for use as prosthetic breast replacements.

13. Products, produced by the process of claim 12, for use as prosthetic breast enhancements.

14. Products, produced by the process of claim 13, for use in conjunction with any allopathic medical procedure.

15. Products, produced by the process of claim 14, not for use with any allopathic medical procedure.
